SEQUENCE LISTING (13 sequences)

SEQ ID NO: 1 - DNA, 33 bp, Artificial
  Sense synthetic oligonucleotide encoding a vascular endothelial growth factor-binding RNA
aacggccgcggctagtccacacacagaaccgtt

SEQ ID NO: 2 - DNA, 39 bp, Artificial
  Anti-sense synthetic oligonucleotide encoding a vascular endothelial growth factor-binding RNA
aacggttctgtgtgtgtggactagccgcggccgtttcga

SEQ ID NO: 3 - DNA, 37 bp, Artificial
  Sense synthetic oligonucleotide encoding a vascular endothelial growth factor-binding RNA
cgcgaacggccgcggctagtccacacacagaaccgtt

SEQ ID NO: 4 - DNA, 39 bp, Artificial; identical to SEQ ID NO: 2
  Anti-sense synthetic oligonucleotide encoding a vascular endothelial growth factor-binding RNA

SEQ ID NO: 5 - DNA, 14 bp, Artificial
  5′ terminal aptamer sequence
gggggggccccccc

SEQ ID NO: 6 - RNA, 14 nt, Artificial
  3′ terminal aptamer sequence
aaaaaaauuuuuuu

SEQ ID NO: 7 - DNA, 32 bp, Artificial
  Synthetic mixed-backbone oligodeoxynucleotide
tggtaccactcgttcccggatggatgctagac

SEQ ID NO: 8 - DNA, 75 bp, Artificial
  Synthetic aptamer sequence with specificity for human L-selectin
ctacctacgatctgactagccggacatgagcgttacaaggtgctaaacgtaacgttgcttactctatgtagttcc

SEQ ID NO: 9 - DNA, 93 bp, Artificial
  Synthetic bi-functional oligodeoxynucleotide
ctacctacgatctgactagccggacatgagcgttacaaggtgctaaacgtaacgttgcttactctatgtagttcctggtaccactcgttcccg

SEQ ID NO: 10 - DNA, 27 bp, Artificial
  Synthetic oligodeoxynucleotide
tgcaggggggggggaactacatgagag

SEQ ID NO: 11 - DNA, 75 bp, Artificial; identical to SEQ ID NO: 8
  Synthetic aptamer with high affinity for human L-selectin

SEQ ID NO: 12 - DNA, 32 bp, Artificial
  Synthetic mixed-backbone antisense oligonucleotide
agagtacatcaaggtggtaccactcgttcccg

SEQ ID NO: 13 - DNA, 46 bp, Artificial
  Synthetic mixed-backbone antisense oligonucleotide
agagtacatcaaggtggtaccactcgttcccggatggatgctagac

(19) United States
(12) Patent Application Publication
(10) Pub. No.: US 2005/0153312 A1
(43) Pub. Date: Jul. 14, 2005

(54) Nucleic acid compositions and methods of introducing nucleic acids into cells

(75) Inventors: Andrew H. Segal, Boston, MA (US); Jeffrey Wilson, Brighton, MA (US)

Correspondence Address: PALMER & DODGE, LLP;KATHLEEN M. WILLIAMS, 111 HUNTINGTON AVENUE, BOSTON, MA, 02199, US

(73) Assignee: Genitrix, LLC

(21) Appl. No.: 10/960,128

(22) Filed: Oct. 7, 2004

Related U.S. Application Data
(62) Division of application No. 09/834,109, filed on Apr. 12, 2001, now abandoned.
Application No. 09/834,109 is a continuation-in-part of application No. 09/120,533, filed on Jul. 22, 1998, now abandoned.
Application No. 09/120,533 is a continuation-in-part of application No. 08/898,094, filed on Jul. 22, 1997, now abandoned.
(60) Provisional application No. 60/022,324, filed on Jul. 24, 1996.

(30) Foreign Application Priority Data
Jul. 22, 1998 (WO) PCT/US98/15130

(51) Int. Cl. C12Q 1/68; C07H 21/04; C12N 15/85

(52) U.S. Cl. 435/6; 435/455; 435/320.1; 435/325; 536/23.2

(57) ABSTRACT

The invention relates to a bifunctional nucleic acid which includes a first nucleic acid which comprises an aptamer bonded to a second nucleic acid that possesses a biological activity (herein referred to as a “biological effector sequence”) and which is not a nucleic acid ligand.

This is a divisional application of U.S. Ser. No. 09/834,109, filed Apr. 12, 2001, which is a continuation-in-part application of U.S. Ser. No. 09/120,533, filed Jul. 22, 1998, pending, which is a continuation-in-part application of U.S. Ser. No. 08/898,094, filed Jul. 22, 1997, which takes priority from U.S. Ser. No. 60/022,324, filed Jul. 24, 1996. The entire teachings of the above application(s) are incorporated herein by reference.

### FIELD OF THE INVENTION

The invention relates to the introduction of exogenous nucleic acid molecules into cells.

### BACKGROUND OF THE INVENTION

Methods for the transfer of nucleic acids into cells are of great utility for both physicians and experimental biologists. For example, genes encoding proteins can be transferred to treat diseases or to facilitate laboratory experiments. Furthermore, “antisense” nucleic acid molecules, which inhibit synthesis of a given polypeptide or polynucleotide by binding to the corresponding template, can be similarly applied, as can polynucleotide enzymes (“ribozymes”).

A number of methods have been developed to effect such transfer. In several such methods, the nucleic acid molecule is physically complexed with a ligand that can bind to a molecule on the surface of the target cells. The entire complex, including the nucleic acid, can then be internalized by the cells via receptor-mediated endocytosis. To date, ligands used have included monoclonal antibodies (see, e.g., Ferkol et al (1993) J Clin Invest 92:2394-400; Buschle et al (1995) Hum Gene Ther 6:753-61), microbe-derived polypeptides (see, e.g., Ding et al (1995) J Biol Chem 270:3667-76), and native ligands for targeted receptors (see, e.g., Sosnowski et al (1996) J Biol Chem 271:33647-53; Christen and Roth (1996) Cancer Gene Ther 3:4-10).

Although nucleic acids have traditionally been conceived of as linear molecules that encode information, it has recently been appreciated that some nucleic acid sequences adopt conformations that allow them to bind to other molecules, such as polypeptides, specifically and with high affinity via non-Watson-Crick interactions. Nucleic acid molecules with this property are known as “nucleic acid ligands”.

Nucleic acid ligands consisting of RNA, single-stranded DNA, and double-stranded DNA have been described. Little is known about the structural features that determine the binding capacity and specificity of such ligands. Nevertheless, it is clear that a proper secondary structure is indispensable. “Secondary structure” refers to stable structural features formed by intrastrand Watson-Crick base pairing. Specific types of secondary structural motifs, defined by their internal juxtapositions of paired and unpaired segments, include, e.g., motifs conventionally known as hairpin loops, symmetric hairpin loops, asymmetric bulged hairpin loops, bulges, and pseudoknots (Schimmel (1989) Cell 58:9-12). The importance of secondary structure to the binding activity of nucleic acid ligands was illustrated, for example, by the studies of Uhlenbeck et al (1983) J Biomolec Structural Dynamics 1:539 and Romaniuk et al (1987) Biochemistry 26:1563.

Nucleic acid ligands have been used to bind soluble molecules and inhibit their biological activities (see, for example, Jellinek et al (1994) Biochemistry 33:10450-56; Bock et al (1992) Nature 355:564-66; Ishizaki et al (1996) Nat Med 2:1386-89). Nucleic acid ligands that bind to cell surface molecules are also possible (Gold and Tuerk, U.S. Pat. No. 5,270,163; Gold and Tuerk, U.S. Pat. No. 5,475, 096), and for the purposes of the invention are termed “aptamers”.

Cell surface molecules are often engaged in signal transduction and cell-cell communication, which is critical for immune responses and tumorigenesis. There are strong interest in understanding the functional pathway of cell surface molecules and whether their function can be modified to modulate immune reactions or tumorigenesis. Nucleic acid ligands that bind to cell surface molecules have promising research and medical utilities. For example, CD40ligand (CD154) is a member of the TNF family of molecules which is expressed on activated T cells. Antibodies to CD154 have been shown to suppress T cell and antibody mediated immune responses in a number of experimental systems. These include inhibition of graft rejection and blocking autoimmune disorders (Durie, F. H. et al. 1993. Science 261:1328). The combined use of anti-CD40ligand antibodies and CD28 blockers (i.e. CTLA-4Ig) has been shown to be effective in blocking graft rejection in both murine and rhesus transplant models (Larsen, C. P. et al. 1996. Nature 381: 434; Kirk, A. D. 1997. Proc. Natl. Acad. Sci. 94:8789). More recently, the use of anti-CD40ligand antibody as a single agent in rhesus kidney allografts has shown that this treatment is remarkably efficacious (Kirk, A. D. et al. 1999. Nature Medicine 5: 686.).

CD40ligand is also expressed on activated platelets and this observation has kindled interest in the role of CD40ligand-CD40 interactions in vascular biology (Henn, V. et al. 1998. Nature 391:591). CD40 and CD40 ligand expression has also been reported on vascular endothelium and smooth muscle cells (Mach, F. et al. 1997. Proc. Natl. Acad. Sci. 94:1931). One report has suggested that inhibition of CD40ligand: CD40 interactions may diminish the development of atherosclerotic lesions (Mach, F. et al. 1998. Nature 394: 200). Atherosclerosis has been viewed as a disease state in which inflammatory processes of the immune system may play a role. Given the potential therapeutic results of inhibiting the activity of the CD40ligand, it would be desirable to have high affinity and high specificity inhibitors of this molecule.

U.S. Pat. No. 6,171,795 teaches the identification and isolation of nucleic acid ligands through their binding affinity to CD40ligand (herein incorporated by reference). The nucleic acids identified may act similarly as the antibody in inhibiting the function of CD40ligand, therefore block graft rejection and autoimmune disorders.

Jeong et al. teaches that an in-vitro selected RNA aptamer against the Ssalyl Lewis X (sLeX) can inhibit cell adhesion, therefore serves as an alternative to a sLeX antibodyin controlling cell adhesion of inflammatory process (2001, Biochemical and Biophysical research Communication 281: 237-243).

It is the object of the invention to provide compositions and methods for introducing nucleic acids, including non-aptamer nucleic acids, into cells using aptamers as ligands. Specifically the invention pertains to bifunctional nucleic acid molecules that comprise an aptamer and another useful nucleic acid sequence that is not an aptamer.

### SUMMARY OF THE INVENTION

One aspect of the invention features nucleic acid molecules, herein referred to as “bifunctional nucleic acid molecules”, which can bind to a cell surface with at least micromolar affinity, and which comprise: a first nucleic acid which comprises an aptamer bonded to a second nucleic acid that possesses a biological activity (herein referred to as a “biological effector sequence”) and which is not an aptamer.

The invention thus relates to a nucleic acid molecule comprising a first nucleic acid sequence comprising an aptamer covalently linked to a second nucleic acid sequence comprising a biological effector sequence.

The invention also relates to a nucleic acid molecule comprising a first nucleic acid sequence comprising an aptamer linked via Watson-Crick base pairing to a second nucleic acid sequence comprising a biological effector sequence.

A molecule of the invention may also further comprise a third nucleic acid sequence which is an aptamer that is covalently linked to the nucleic acid molecule.

A molecule of the invention may also further comprise a third nucleic acid sequence which is an aptamer that is linked via Watson-Crick base pairing to the nucleic acid molecule.

In preferred embodiments, the third nucleic acid sequence comprises an aptamer that is different from the first nucleic acid sequence comprising an aptamer.

Preferably, a molecule of the invention comprises DNA or RNA.

The phrase “nucleic acid molecule” as used herein is intended to include structures that comprise nucleotides covalently bound to each other to form polymers that can, for example, be linear, cyclic, and/or branched. The bonds between sequential nucleotides in the polymer are referred to herein as a “backbone” of the molecule and can be, for example, phosphodiester, phosphorothioate, phosphoramidate, thioformacetal, carboxamide, methylphosphonate, or peptide bonds. The molecules can be single-stranded and/or multi-stranded, but in a preferred embodiment are single- or double-stranded. According to the invention, a nucleic acid molecule can include naturally occurring and non-naturally occurring nucleotides, and can include non-nucleotide structures, e.g. amino acids, carbohydrates, and metal ions. The nucleotides may be chemically substituted at the ribose and/or phosphate and/or base positions. For example, the nucleotides can incorporate 5-pyr, 2′-amino, 2′-fluoro, or 2′-O-methyl groups. A nucleic acid molecule can include structures that are linked to each other by means other than covalent bonds, e.g. by Watson-Crick base pairing between overlapping complementary sequences.

An “aptamer” is a nucleic acid molecule that is capable, by virtue of secondary and/or tertiary structure, of binding to a cell surface molecule such as a carbohydrate or a protein in a selective fashion, preferably with an affinity as strong as in the micromolar range (1-100 uM) and more preferably with an affinity even stronger, e.g., in the nanomolar to picomolar range (1-100 nM affinity and 1-100 pM affinity). That is, the aptamer will selectively bind to the target molecule or cell with an affinity that is at least 10-fold greater affinity than the affinity with which the aptamer binds to a non-target molecule. In general, an aptamer will comprise about 10-400 nucleotides, more preferably 20-100, and most preferably 25-50. “Selective” binding refers to specific binding to a target cell surface molecule but not to most other molecules on the cell surface or on other cells that do not contain the targeted cell surface molecule.

Preferably, the aptamer and the biological effector sequence are linked via a covalent bond that is a phosphodiester bond.

Where the aptamer and the biological effector sequence share a continuous backbone, a nucleotide sequence of the bifunctional nucleic acid molecule can contribute to the biologic function of both.

In other preferred embodiments, the aptamer may be 3′ to the biological effector sequence or 5′ to the biological effector sequence. The aptamer and the biological effector sequence can be linked in a 3′-3′ manner, e.g. via an acetal group. A bifunctional nucleic acid molecule can comprise multiple aptamers and/or biological effector sequences. In a preferred embodiment, the aptamer and biological effector sequence are admixed with a polycation, such as poly-L-lysine. In yet another preferred embodiment, the aptamer and the biological effector sequence are linked via a biotin-streptavidin interaction.

It is preferred that the aptamer of a bifunctional nucleic acid molecule be able to bind to a cell-surface polypeptide present on a cell, preferably with nanomolar to picomolar (1 pM-100 nM), preferably 10 pM-50 nM, most preferably 50 pM-10 nM) affinity.

In one embodiment, the biological effector sequence of a bifunctional nucleic acid molecule comprises a sequence that encodes a biologically or therapeutically useful polypeptide or polynucleotide. In a preferred embodiment, the sequence is double-stranded DNA and comprises a promoter that is operably linked to the coding sequence. In another preferred embodiment, the biological effector sequence is an mRNA, that is an RNA that translates to a polypeptide, which preferably comprises a 5′ cap and a 3′ poly-A sequence.

In another embodiment, the biological effector sequence comprises a sequence that is antisense to a nucleic acid that is present in the target cell. In preferred embodiments the antisense sequence can either 1) inhibit transcription of the target, or 2) inhibit reverse transcription of the target, or 3) inhibit translation of the target, or 4) inhibit replication of the target, or 5) inhibit the target from assuming a functional conformation.

In other preferred embodiments, the antisense sequence is complementary to at least five contiguous nucleotides of the target, or is complementary to at least seven contiguous nucleotides of the target, or is complementary to at least ten contiguous nucleotides of the target, or is complementary to at least fifteen contiguous nucleotides of the target, or is complementary to at least twenty contiguous nucleotides of the target. Thus, the antisense sequence may form a Watson-Crick base-paired hybrid with a target RNA or DNA and inhibit expression (translation or transcription or regulatory binding) to the target molecule.

In another preferred embodiment, the biological effector sequence comprises a nucleic acid enzyme, e.g. a ribozyme.

In one embodiment, a bifunctional nucleic acid molecule can bind more than one cell surface molecule at once via its aptamers. In a preferred embodiment, the aptamer can concatimerize and comprises first and second unpaired nucleic acids, each of which is complementary to itself; however, neither of which is complementary to the other. This structure allows concatemers of the aptamer to form and incorporate into the bifunctional nucleic acid molecule. A concatemer is at least two aptamers, and up to 1000 or even more (10,000) aptamers, but is preferably in the range of two-500 aptamers, and most preferably in the range of two- 50 aptamers. It is believed that such concatemers can cross-link the target receptors, increasing endocytosis.

The invention also pertains to vectors which include the bifunctional nucleic acid molecules of the invention, cells which into which have been introduced such vectors, and cells into which have been introduced the molecules of the invention.

In another aspect, the invention pertains to nucleic acid molecules that can be used in the production of bifunctional nucleic acid molecules. In a preferred embodiment, these molecules are DNA or RNA molecules that can serve as templates for the assembly of bifunctional nucleic acid molecules, e.g. by possessing Watson-Crick complementarity to a bifunctional nucleic acid molecule and by directing the assembly of a complementary nucleic acid, e.g. by the action of a nucleotide polymerase.

In another aspect, the invention pertains to compositions that include the nucleic acid molecules of the invention and that include substances that can facilitate the use of these nucleic acid molecules in vivo and/or in vitro. In one embodiment, the composition includes a pharmaceutically acceptable carrier.

In another embodiment, the composition includes a substance that improves the uptake of exogenous polynucleotides by cells, e.g. by inducing endocytosis and/or phagocytosis and/or macropinocytosis and/or micropinocytosis and/or potocytosis. In yet another embodiment, such compositions can include substances that disrupt intracellular vesicles containing the nucleic acid molecules of the invention.

It is preferred that a composition of the invention comprise a bifunctional nucleic acid molecule and a substance that facilitates the intracellular trafficking of the biological effector sequence to its site of action. Such substances can include, for example, those that facilitate release from an intracellular vesicle or those that facilitate translocation to the nucleus.

In yet another aspect, the invention pertains to methods of introducing biological effector sequences into cells.

In a preferred embodiment, the complexes of the invention are admixed in vitro with cells bearing the receptor for the aptamer. It is further preferred that the cells are subjected to transfection with the complexes utilizing, e.g., calcium phosphate precipitation or electroporation. In another preferred embodiment, the cells are treated with an endosomal disruption agent. In another embodiment, the complexes of the invention are administered to an organism, e.g. an animal, a mammal, e.g. a primate, e.g. a human.

In another aspect, the invention features a method of introducing a biological effector sequence into an organism, comprising introducing a biological effector sequence into a cell by contacting the bifunctional nucleic acid with a host cell, and administering the cell to an organism.

Other features and advantages of the invention are described hereinbelow and in the claims.

### DETAILED DESCRIPTION OF THE INVENTION

The invention is based on the recognition that a bifunctional nucleic acid, that is, a first nucleic acid sequence which is an aptamer specific for a cell surface molecule such as a protein or carbohydrate that is bound to a second nucleic acid sequence that is a biological effector sequence and not an aptamer can facilitate entry of the second nucleic acid sequence into a target cell. The bifunctional nucleic acid molecule is composed of two nucleic acid sequences or fragments that are bound together via covalently bonding or via Watson-Crick base-pairing along a portion of the two sequences, the portion being long enough to provide sufficient stability for the two molecules to form a hybrid and to facilitate entry of the second sequence into the target cell.

The contents of citations referred to herein are incorporated by reference in their entirety.

Production of Aptamers

An aptamer specific for a given target molecule is isolated from a heterogeneous library of polynucleotides by affinity purification against the target molecules. For example, in the “SELEX” procedure described in Gold and Tuerk, U.S. Pat. No. 5,270,163, the following steps are performed:
1) The library is prepared as follows. For example, a random collection of oligonucleotides can be generated by programming an automated synthesizer to select a solution of multiple bases for incorporation at one or more steps. Ideally, this library contains at least about 10⁹ nucleic acids, and no more than about 10¹⁸. If an RNA library is desired, it may be convenient to first obtain a corresponding DNA library and then, after cloning into a suitable plasmid, obtain the RNA by in vitro transcription.
2) The library is contacted with the target molecule under physiological chemical conditions. Contacting can occur either with ligands and target free in solution, or with the target bound to a support. Examples of supports include agarose or sepharose matrices. The target can be coupled to such matrices either covalently or noncovalently.
3) The candidate aptamers with the highest affinities for the target (e.g., 5-50% of the polynucleotides) are then partitioned from those with lower affinities. In one partitioning method, the target is a polypeptide and the candidate/target mixture is passed through a nitrocellulose membrane and the membrane washed with, e.g., 200 mM potassium acetate, 50 mM Tris-HCl pH 7.7. This often elutes free candidates (“losers”) while leaving aptamer/target complexes bound. The “winners” are then released from the target by washing the membrane with, e.g., 200 ul 7M urea, 20 mM Na citrate (pH 5.0), 1 mM EDTA with 500 ul phenol (equilibrated with 0.1M Na acetate pH 5.2).

In another partitioning method (Toole et al, U.S. Pat. No. 5,582,981), free candidates are eluted from a support-bound target using a suitable buffer. The target is then uncoupled from the support and the aptamer/target complexes collected. The nucleic acid molecules are isolated by standard denaturation techniques, such as phenol extraction.

If either of the above partitioning methods are used for a target that is a cell surface molecule, it is preferred that only the extracellular portion of the molecule be employed in the assay.

In a partitioning method that is useful for cell surface targets, cells are obtained that do not express the desired target. The library is admixed with the cells, and the unbound candidates are collected. These candidates are then admixed with cells that are identical to those above, except that they do express the target. For example, they may be transfected with a gene encoding a polypeptide target, or enzymatically modified to bear a carbohydrate target. The cells are spun down and the unbound candidates in the supernatant discarded. The cells are then treated with phenol to release the aptamers.
4) The selected polynucleotides are amplified, typically by PCR. If the candidates are RNA, they are reverse transcribed, the DNA is amplified, and RNA is again generated by reverse transcription as above. The product is a library enriched for capacity to bind the target.
5) Steps 2-5 are repeated. In each round, binding and/or partitioning conditions can be made more stringent. At least ten rounds are usually required for optimal results.

Cells that can bear targets for aptamers include bacterial, fungal, plant, yeast and mammalian cells, e.g., malignant tumor cells, fibroblasts, endothelial cells, epithelial cells including respiratory epithelial cells, pluripotent stem cells, leukocytes, endocrine cells such as islet cells, hepatocytes, keratinocytes, melanocytes, pericytes, germ cells, neurons, myocytes including cardiac myocytes, osteocytes, osteoblasts and chondrocytes.

Cell surface molecules that are candidate targets for aptamers include, e.g., the transferrin receptor, the asialoglycoprotein receptor, the TSH receptor, FGF receptors, CD3, the IL-2 receptor, the growth hormone receptor, the insulin receptor, the acetylcholine receptor, adrenergic receptors, VEGF receptors, and receptors for viruses such as the adenovirus receptor.

Aptamers useful according to the invention include but are not limited to aptamers specific for the human MDR1 and MRP genes, including SEQ ID Nos: 67-117, SEQ ID Nos: 129-179 and 185-196, SEQ ID Nos: 199-235, SEQ ID Nos: 251-290, SEQ ID Nos: 293-384 of WO 96/40715; aptamers specific for insulin receptor antibody, including SEQ ID Nos: 4-15 of WO 95/30775; aptamers specific for peripheral blood mononuclear cells, including SEQ ID Nos: 7-39 of WO 96/34874; aptamers specific for multidrug resistance (MDR) phenotype, including SEQ ID Nos: 88091 of WO 96/40715; aptamers specific for TNFα, including SEQ ID Nos: 209-255 of WO 96/40717; and aptamers specific for cytokines, including those disclosed in WO 96/40717.

Production of Nucleic Acid Molecules

In one aspect, the invention pertains to nucleic acid molecules that can be used in the production of bifunctional nucleic acid molecules. In a preferred embodiment, these molecules are DNA or RNA molecules that can serve as templates for the assembly of bifunctional nucleic acid molecules, e.g. by possessing Watson-Crick complementarity to a bifunctional nucleic acid molecule and by directing the assembly of a complementary nucleic acid, e.g. by the action of a nucleotide polymerase. Such molecules are referred to herein as “bifunctional nucleic acid molecule-encoding sequences”, and are useful for producing bifunctional nucleic acid molecules in which an aptamer and a biological effector sequence share a continuous backbone. Bifunctional nucleic acid molecule-encoding sequences can include sequences that are not Watson-Crick complementary to a bifunctional nucleic acid molecule, e.g. sequences that regulate and/or direct the activity of nucleotide polymerases, e.g. promoter sequences.

Another aspect of the invention pertains to cloning vectors containing bifunctional nucleic acid molecules or bifunctional nucleic acid molecule-encoding sequences. As used herein, the term “cloning vector” refers to a nucleic acid molecule capable of transporting another nucleic acid molecule to which it has been linked. One type of vector is a “plasmid”, which refers to a circular, double-stranded nucleic acid loop into which additional nucleic acid segments can be ligated. Another type of vector is a viral vector, wherein additional nucleic acid segments can be ligated into the viral genome. Cloning vectors can include one or more nucleic acid sequences that can regulate biochemical processes by cis-acting mechanisms, e.g. promoters and/or binding sites for template-directed nucleotide polymerases. Examples of cloning vectors include pBR322 (Watson (1988) Gene 70:399-403), pUC19 (Yanisch-Perron et al (1985) Gene 33:103-19), and pBK-CMV and pBK-RSV (both available from Stratagene, La Jolla, Calif.).

The recombinant cloning vectors of the invention comprise a nucleic acid of the invention in a form useful for producing larger quantities of a molecule of the invention than are available prior to operations on the vector in vivo or in vitro. For example, a bacterial cloning plasmid containing a bifunctional nucleic acid molecule sequence or a bifunctional nucleic acid molecule-encoding sequence can be used to amplify the number of copies of the sequence by introduction of the plasmid into a suitable bacterial host. Another type of cloning vector according to the invention is a plasmid which comprises: 1) a template for a bifunctional nucleic acid molecule, which bifunctional nucleic acid molecule comprises an RNA aptamer and an RNA biological effector sequence linked via a phosphodiester bond; and 2) cis-acting sequences sufficient to allow in vitro transcription by a suitable RNA polymerase, e.g. T7 polymerase. Recombinant cloning vectors of the invention can also include vectors that lack a biological effector sequence and a biological effector-sequence encoding sequence but include: 1) an aptamer or an aptamer-encoding sequence and 2) a site, e.g. a multiple cloning site, which can facilitate the insertion of a biological effector sequence or a biological effector-sequence encoding sequence in such a manner that the resulting sequence is, or can serve as a template for, a bifunctional nucleic acid molecule in which the aptamer and the biological effector sequence are joined by a phosphodiester bond.

Numerous methods for the synthesis of nucleic acid sequences, including aptamers and biological effector sequences, are available and can be applied to the production of bifunctional nucleic acid molecules. For example, RNA molecules can be produced by in vitro transcription. In this technique, a gene encoding the desired RNA is cloned into a plasmid so that the gene is operably linked to a promoter for a DNA-dependent RNA polymerase, e.g., T7 RNA polymerase. The plasmid is then admixed, in a suitable buffer, with the RNA polymerase under conditions such that transcription occurs.

The polymerase chain reaction can be used to synthesize a specific DNA sequence (Mullis, U.S. Pat. No. 4,683,202). A double-stranded template sequence is admixed with a thermophilic DNA-dependent DNA polymerase, excess 5′ and 3′ primers, and excess free nucleotides in a suitable buffer. The mixture is subjected to repeated thermal cycling in a manner that allows melting and reannealing of complementary sequences and coordinated activation and deactivation of the polymerase.

Nucleic acid molecules can also be synthesized by machines that execute chemical polymerization of nucleotides. One such machine is produced by Applied Biosystems of Foster City, Calif.

Biological Effector Sequences Useful According to the Invention

Examples of nucleic acid sequences that can serve as biological effector sequences include sequences that encode useful polypeptides or useful polynucleotides, sequences that are “antisense” to nucleic acids that are present in the target cells, nucleic acid enzymes (e.g. ribozymes), nucleic acid sequences that can regulate biochemical processes by cis-acting mechanisms (e.g—9-promoters or ribosome binding sites), and other useful nucleic acid sequences. A sequence is “antisense” if it is Watson-Crick complementary to and can inhibit the biological function of another nucleic acid molecule.

Examples of coding sequences that are useful according to the invention include, but are not limited to, sequences encoding the cystic fibrosis transmembrane regulator (Genbank Accession No. M28668), which is useful for treating cystic fibrosis; Factor VIII (Genbank Acc. No. E00527), which is useful for treating hemophilia; hemoglobin beta chain (Genbank Acc. No. V00497), which is useful for treating thalassemias; alpha-1-antitrypsin (AAT) (Genbank Acc. No. E00195), which is useful for treating AAT deficiency; insulin (Genbank Acc. No. J00265), which is useful for treating type 1 diabetes mellitus; TGF-beta (Genbank Acc. No. X02812 J05114), which is useful for treating inflammation and atherosclerosis; conditionally toxic polypeptides such as herpes simplex virus thymidine kinase (Genbank Acc. No. V00470), which is useful for treating malignant tumors; and antisense, ribozyme, and nucleic acid ligand molecules.

Examples of nucleic acid sequences that possess enzymatic activity and are useful according to the invention include, but are not limited to, ribozymes directed at mRNAs for proteins such as N-ras (Scherr et al (1997) J Biol Chem 272:14304-13) and c-fos (Scanlon et al (1994) Proc Natl Acad Sci USA 91:11123-27), which are useful for treating malignant tumors, and ribozymes directed at molecules involved in the life cycle of viruses such as HIV (Zhang et al (1996) Biochem Biophys Res Commun 229:466-71; Raillard and Joyce (1996) Biochemistry 35:11693-701; Michienza et al (1996) Proc Natl Acad Sci USA 93:7219-24), influenza A virus (Tang et al (1994) J Med Virol 42:385-95), and Tobacco Mosaic Virus (de Feyter et al (1996) Mol Gen Genet 250:329-38), which are useful for treating the respective viruses. Other examples of nucleic acid sequences that possess enzymatic activity and are useful according to the invention include, but are not limited to, hairpin ribozymes; hammerhead ribozymes; Group I introns; Group II introns; ribozymes directed at molecules involved in the life cycle of viruses such as hepatitis B virus, hepatitis C virus, hepatitis D virus, hepatitis E virus, foot and mouth disease virus, mouse hepatitis virus, Moloney murine leukemia virus, bovine leukemia virus, influenza B virus, herpes viruses, lymphocytic choriomeningitis virus, potato leafroll virus, arenaviruses, potato virus YN, respiratory syncytial virus, retroviruses, rhabdoviruses, hemorrhagic fever agents, coronaviruses, rhinoviruses, myxoviruses, paramyxoviruses, and arboviruses; ribozymes directed at integrin mRNAs; RNase P; hepatitis D virus ribozymes; ribozymes directed at beta amyloid protein precursor mRNA; ribozymes directed at alpha actin; Tetrahymena ribozymes; ribozymes directed at phosphorothionate bonds; ribozymes directed at, oncogenes such as H-ras, c-fos, c-myc, PML/RAR alpha bcr/abl, p53, AML1/MTG8, lck, fyn; ribozymes directed at thymidylate synthetase mRNA; ribozymes directed at mdr-1 mRNA; ribozymes directed at mdr-2 mRNA; ribozymes directed at components of telomerase; ribozymes directed at GAP-43 mRNA; ribozymes derived from chicory yellow mottle virus; ribozymes derived from arabis mosaic virus; ribozymes directed at amelogenin mRNAs; ribozymes directed at serum amyloid A mRNAs; ribozymes directed at osteopontin mRNA; polynucleotide kinase ribozymes; ribozymes directed at lipoxygenase mRNAs; ribozymes directed at cytokine mRNAs; at ribozymes directed at NF-1L6 mRNAs; RNA ligase ribozymes; DNA ligase ribozymes; ribozymes directed at alkaline phosphatase mRNAs; Neurospora VS ribozymes; DNA enzymes such as DNA metalloenzyme DNA ligases; ribozymes directed at beta-glueuronidase mRNAs; ribozymes directed at plasminogen activator inhibitor mRNAs; self-alkylating ribozymes; ribozymes directed at glucose-regulated protein mRNAs; ribozymes directed at yeast ADE1 gene mRNA; ribozymes directed at insulin receptor substrate mRNAs; RNA cyclase ribozymes; -ribozymes directed at glucokinase mRNAs; ribozymes directed at Nramp mRNAs; ribozymes directed at aberrant immunoglobulin light chain mRNAs; avocado sunblotch ribozymes; ribozymes directed at npt mRNAs; Pneumocystis carinii ribozymes; ribozymes directed at chloramphenicol acetyltransferase mRNAs; ribozymes directed at acetyl CoA carboxylase mRNAs; trans-splicing ribozymes; ribozymes directed at platelet-derived growth factor mRNAs; ribozymes directed at tumor necrosis factor alpha mRNAs; ribozymes directed at urokinase receptor mRNAs; ribozymes directed at atrial natriuretic factor mRNAs; ribozymes directed at pleiotrophin mRNAs; ribozymes directed at beta-2 microglobulin mRNAs; DNA-cleaving ribozymes; ribozymes directed at coliphages; Didydium ribozymes; chloroplast ribozymes; RNA polymerase ribozymes; DNA polymerase ribozymes; ribozymes directed at Drosophila white mRNAs; ribozymes that hydrolyze phosphoric acid; ribozymes directed at calretinin mRNAs; ribozymes directed at VEGF mRNAs; integrase ribozymes; sunY ribozymes; ribozymes directed at methyltransferase mRNAs; peptide cleaving ribozymes; peptide synthesizing ribozyme; ribozymes directed at prion mRNAs; ribozymes directed at inhibin mRNAs; self-copying ribozymes; ribozymes directed at alpha lactalbumin mRNAs; aminoacyl esterase mRNAs; tobacco ringspot virus ribozymes; barley yellow dwarf virus ribozymes; Chlamydomonas ribozymes; ribozymes that can specifically cleave single-stranded DNA; ribozymes directed at histone mRNAs, and ribozymes directed at glucan branching enzyme mRNAs.

Examples of antisense nucleic acids that are useful according to the invention include, but are not limited to, molecules that are antisense to genes for K-ras (Alemany et al (1996) Cancer Gene her 3:296-301), c-kit (Yamanishi et al (1996) Jpn J Cancer Res 87:534-42), and c-myc (Leonetti et al (1996) J Nail Cancer Inst 88:419-29), useful for treating malignant tumors; HIV tat (Biasalo et al (1996) J Virol 70:2154-61), which is useful for treating which is useful for treating HIV infection; Staphylococcus aureus alpha-toxin (Kemodle et al (1997) Infect Immun 65:179-84), which is useful for treating S. aureus infection; TYLCV Rep protein (Bendahmane and Gronenborne (1997) Plant Mol Biol 33 :351-57), which is useful for treating TYCLV infection; and Hepatitis C core protein (Moradpour et al(1996) Virology 222:51-63) which is useful for treating Hepatitis C infection. Other examples of antisense molecules useful according to the invention include those that have as their targets nucleic acids encoding gastrin, urokinase, Ha-ras, reverse transcriptase, HIV TAR, HIV gag, HIV rev, HIV nef, mycobacterial proteins, Tobacco Mosaic Virus proteins, hepatitis B virus proteins, hepatitis C virus proteins, hepatitis D virus proteins, hepatitis E virus proteins, Hepatitis G virus proteins, foot and mouth disease virus proteins, mouse hepatitis virus proteins, Moloney murine leukemia virus proteins, bovine leukemia virus proteins, influenza A virus proteins, influenza B virus proteins, herpes virus proteins, lymphocytic choriomeningitis virus proteins, potato leafroll virus proteins, arenavirus proteins, potato virus YN proteins, respiratory syncytial virus proteins, retrovirus proteins, rhabdovirus proteins, hemorrhagic fever agent proteins, coronavirus proteins, rhinovirus proteins, myxovirus proteins, paramyxovirus proteins, and arbovirus proteins, integrins, RNase P, beta amyloid protein precursor, alpha actin, ribozymes directed at oncogenes such as c-fos, c-myc, PML/RAR alpha bcr/abl, p53, AML1/MTG8, lck, fyn thymidylate synthetase, mdr-1, mdr-2, components of telomerase, GAP-43, chicory yellow mottle virus proteins, arabis mosaic virus proteins, amelogenin, serum amyloid A mRNAs, osteopontin, lipoxygenase, cytokines mRNAs, NF-IL6, DNA ligase ribozymes, alkaline phosphatase, beta-glucuronidase, plasminogen activator inhibitor, glucose-regulated proteins mRNAs, insulin receptor substrate mRNAs, glucokinase, Nramp, immunoglobulin light or heavy chains, T cell receptor chains, Pneumocystis carinii proteins, chloramphenicol acetyltransferase, acetyl CoA carboxylase, platelet-derived growth factor, tumor necrosis factor alpha, urokinase receptor, atrial natriuretic factor, pleiotrophin, beta-2 microglobulin, calretinin, VEGF mRNAs, integrase, methyltransferase, prion proteins, inhibin, alpha lactalbumin, aminoacyl esterase, tobacco ringspot virus proteins, barley yellow dwarf virus proteins, Chlamydomonas proteins, histone proteins, glucan branching enzyme, bacterial proteins, fungal proteins, rickettsial proteins, proteins involved in autoimmune diseases, mRNAs, tRNAs, nucleoprotein RNAs, and snRNAs.

Assembly of Bifunctional Nucleic Acid Molecules

Aptamers and biological effector sequences cm be linked using many methods. For example, if an aptamer and a biological effector sequence comprise mutually complementary sequences, they can be annealed to each other. The length of the mutually complementary sequences in each of the aptamer and biological effector sequence will be sufficiently long to permit the two molecules to form a hybrid that will not dissociate under the following conditions: at least 15′C, pH 7.4, 125 mM NaCl in physiological compatible buffer. This length typically is in the range of 8-50 base pairs, preferably 10-20 base pairs, most preferably, 10-15 base pairs. In order to determine if the length of mutual complementarity is sufficient, a functional assay can be performed in which the hybrid molecule is used to transfect a target cell for which the aptamer-portion of the bifunctional nucleic acid is specific (in terms of its selectively recognizing a cell surface molecule on the target cell); if the biological effector sequence is detected in the cell (either its direct presence or detection of a product or activity encoded by the effector sequence) at a level that is at least 20% greater and preferably 50%-100% or more than a comparative transfection using the biological effector sequence alone (i.e., without the Watson-Crick base-paired aptamer or with an aptamer that is not base-paired or covalently bonded to the effector sequence), then the length of mutual complementarity is considered sufficient according to the invention.

Typically, the aptamer and biological effector fragments are admixed in a suitable buffer and heated to approximately 90° C. The mixture is then allowed to cool to a temperature that allows Watson-Crick base pairing between the complementary sequences. The temperature below which this occurs can be estimated by adding together the temperature values of each base of one of the complementary sequences, viz. 2° C. for A or T and 4° C. for G or C. If it is desired to produce a bifunctional nucleic acid molecule that comprises concatemers of aptamers and/or biological effector sequences formed by Watson-Crick base pairing, the ratio of each component in the bifunctional nucleic acid molecule can be manipulated by adjusting the molar ratios of the nucleic acid molecules in the annealing reaction. Furthermore, the average length of the concatemers can be manipulated by adding, in appropriate molar ratio, a “termination sequence”. A termination sequence comprises a nucleotide sequence that allows incorporation into a concatemerized bifunctional nucleic acid molecule via Watson-Crick base pairing, but does not comprise a second sequence that would allow further concatemerization.

Another method of assembling a bifunctional nucleic acid molecule involves admixing the aptamer and the biological effector sequence with a polycation, e.g. a polyamine, e.g. polylysine. This is particularly useful for assembling noncovalently linked DNA sequences, since the polyanionic charge of the DNA allows assembly via electrostatic attraction.

Another approach to assembling bifunctional nucleic acid molecules exploits ligase enzymes to covalently link an aptamer and a biological effector sequence. Ligases catalyze the formation of a phosphodiester bond between two nucleic acid molecules. For example, T4 DNA ligase uses as its substrates duplex DNA or RNA, or DNA/RNA hybrids. T4 RNA ligase uses single stranded DNA or RNA. E. coli DNA ligase acts on duplex DNA molecules which have compatible cohesive ends.

### Assay for Gene Transfer

The bifunctional nucleic acid is assayed for its ability to transfer genes into a target cell. For studies aimed at determining transfection efficiency, the biological effector sequence of the bifunctional nucleic acid contains a marker gene for firefly luciferase. For pharmaceutical applications, the bifunctional nucleic acid contains a gene whose expression will have a beneficial therapeutic effect. The bifunctional nucleic acid is incubated with the cells. After incubation, the cells are lysed and are assayed for gene expression. In the case of the luciferase reporter, luciferin and ATP are added to lysed cells and the light emitted is measured with a luminometer.

Cells are harvested on the day of assay by centrifugation at 1200 rpm for 5 mm at room temperature. The cell pellet is resuspended in phosphate buffered saline and re-centrifuged. This operation is performed twice. The cell pellet is then suspended in RPMI 1640 (Gibco Ltd.) to make up a suspension of 2.7×10⁶ cells per ml. The cells are then aliquoted into tubes and 0.75 ml of RPMI medium added, followed by 0.04-0.08 ml of 1 mM FP peptide and finally 0.25 ml of DNA solution. The transfection is then allowed to proceed by incubating the cells at 37° C. for 4 h. After this time, the cells are harvested by centrifugation at 2000 rpm. The cells are then suspended in 1 ml of RPMI and re-centrifuged. Finally, the cells are suspended in 0.5 ml RPMI containing 0.1% fetal bovine serum. At this stage, if desired, the cells are electroporated at 300 V and 250 μF using conventional electroporation.

Each 0.5 ml of transfected cell suspension is transferred to a well of a 12 well plastic culture plate containing 1.5 ml of RPMI 10% FBS. The original transfection tube is rinsed with a further 1 ml of medium and the wash transferred to the culture dish making a final volume of 3 ml. The culture plate is then incubated at 37° C. for 24-72 h in an atmosphere of 5% CO₂. The contents of each well in the culture dish are transferred to centrifuge tubes and the cells collected by centrifugation at 13,000 rpm. The pellet is resuspended in 0.12 ml of Lysis Buffer (100 mM sodium phosphate, pH 7.8; 8 MM MgCl₂, 1 mM EDTA; 1% Triton X-100 and 15% glycerol) and agitated with a pipette. The lysate is centrifuged at 13,000 rpm for 1 minute and the supernatant collected. 80 μl of the supernatant are transferred to a luminometer tube. The luciferase activity is then assayed using a Berthold Lumat L9501 luminometer. The assay buffer used is Lysis buffer containing 10 mM Luciferin and 100 mM ATP. Light produced by the luciferase is integrated over 4 sec and is described as relative light units (RLU) The data are converted to RLU/ml of lysate, RLU/cell or RLU/mg protein (protein concentration of the lysate having been determined in this case by the BioRAD Lowry assay).

Use of this type of pharmaceutical composition in vivo or ex vivo with nucleic acid containing a gene of physiological importance, such as replacement of a defective gene or an additional potentially beneficial gene function, is expected to confer long term genetic modification of the cells and be effective in the treatment of disease.

For example, a patient that is subject to a viral or genetic disease may be treated in accordance with the invention via in vivo or ex vivo methods. For example in vivo treatments, a bifunctional nucleic acid of the invention can be administered to the patient, preferably in a biologically compatible solution or a pharmaceutically acceptable vehicle, by ingestion, injection, inhalation or any number of other methods. The dosages administered will vary from patient to patient; a “therapeutically effective dose” will be determined by the level of enhancement of function of the transferred genetic material balanced against any risk or deleterious side effects. Monitoring levels of gene introduction, gene expression and/or the presence or levels of the encoded anti-viral protein will assist in selecting and adjusting the dosages administered. Generally, a composition including a bifunctional nucleic acid will be administered in a single dose in the range of 10 ng-100 ug/kg body weight, preferably in the range of 100 ng -10 ug/kg body weight, such that at least one copy of the therapeutic gene is delivered to each target cell. The therapeutic gene will, of course, be associated with appropriate regulatory sequences for expression of the gene in the target cell.

Ex vivo treatment is also contemplated within the present invention. Cell populations can be removed from the patient or otherwise provided, transduced with a therapeutic gene in accordance with the invention, then reintroduced into the patient. In general, ex vivo cell dosages will be determined according to the desired therapeutic effect balanced against any deleterious side-effects. Such dosages will usually be in the range of 10⁵-10⁸ cells per patient, daily weekly, or intermittently; preferably 10⁶-10⁷ cells per patient.

A bifunctional nucleic acid according to the invention may be used to treat X-linked γ-globulinemia. The bifunctional nucleic acid will contain the Bruton's tyrosine kinase gene (Vetrie et al., 1993, Nature 361:226-233), which is carried on a 2.1 kb fragment delineated by the PvuI site at position (+33) and the HindIII site at position (+2126). The therapeutic gene may also encode a splice site and poly A tail, which may include portions of the human β globin locus splice and poly A signals; i.e., a BamHIXbaI 2.8 kb 3′ splice/poly A flanking sequence containing exon 2 IVSII—exon 3—polyA sequences.

A bifunctional nucleic acid containing the Bruton's tyrosine kinase gene is assembled as described herein and used to treat X-linked γ-globulinemia by introducing the bifunctional nucleic acid directly into a patient for in vivo gene therapy or contact the bifunctional nucleic acid with pre-B cells for ex vivo therapy, as described in Martensson et al.; Eur. Jour. Immunol. 1987, 17:1499; Okabe et al., Eur. Jour. Immunol. 1992, 22:37; and Baneiji et al., Cell 33:729, 1983, and administering the transfected pre-B cells into a patient afflicted with X-linked γ-globulinemia. A bifunctional nucleic acid for treatment of X-linked γ-globulinemia will include a ligand for targeting of a preB cell. Such ligands are well-known in the art and will be specific for and capable of targeting one or more of the following cell surface markers: CD9, CD 10, CD 19, CD20, CD22, CD24, CD38, CD40, CD72, and CD74.

A bifunctional nucleic acid described herein also may be used for treatment of Gaucher's disease. Gaucher's disease stems from one of two different genetic mutations. Gaucher's type 1 is a CGG→CAG mutation, which results in an Arg→Gln substitution at position 119 of the :β-glucocerebrosidase polypeptide (Graves, DNA 7:521, 1988). Gaucher's type 2 is a CTG→CCG mutation, which results in a Leu→Pro substitution at position 444 of the Z-glucocerebrosidase polypeptide (Tsuji, NEJM 316:570, 1987). The presence of a :β-glucocerebrosidase gene encoding a wild type polypeptide is believed to substantially correct Gaucher's disease. Therefore, a therapeutic bifunctional nucleic acid useful according to the invention includes the β-glucocerebrosidase gene, as described in Horowitz et al., 1989, Genomics 4:87-96, which is carried, as disclosed in Horowitz et al., on a 9722 base pair fragment extending from a BamHI site in exon 1 to an EcoRV site 31 to polyadenylation site. This fragment contains 11 exons and all intervening sequences, with translational start in exon 2. Sequences conferring position-independent and tissue-specific gene expression may be included in the construct and are carried on an 11.8 kb XhoI-SacI fragment from pIII.lyx construct as described in Bonifer et al., 1990, Euro. Mol. Biol. Org. Jour. 9;2843.

A bifunctional nucleic acid containing the β-glucocerebrosidase gene is assembled as described herein and used to treat Gaucher's disease by introducing the bifunctional nucleic acid directly into the host for in vivo treatment, or into isolated macrophages for ex vivo therapy, as described in Immunology and Cell Biology, 1993, Vol. 71, pages 75-78 and introducing the transfected macrophages into a patient afflicted with Gaucher's disease. Expression of the wild type transgene in a patient afflicted with Gaucher's disease should result in correction of the diseased state. The bifunctional nucleic acid will contain an aptamer that specifically targets a cell surface antigen on a macrophage. Such aptamers may be easily prepared from procedures well-known in the art and described hereinabove, for example, aptamers having specificity for and capable of targeting one or more of the following cell surface markers: CD14, CD16, CD26, CD31, CDw32, CD36, CD45RO, CD45RB, CD63, CD71, CD74, CD23, CD25 and CD69.

The cells targeted for in vivo or ex vivo gene transfer in accordance with the invention include any cells to which the delivery of the therapeutic gene is desired. Such cells will bear a cell surface marker for which a corresponding specific ligand is available or can be prepared to allow for cell-specific targeting according to the invention. For example, cells of the immune system such as T-cells, B-cells, and macrophages, hematopoietic cells, and dendritic cells, each cell of which bears one or more well-known cell surface receptors having corresponding aptamers which may be selected for use as a targeting ligand in the bifunctional nucleic acid of the invention, depending upon the selected cell. Using established technologies, stem cells may be used for gene transfer after enrichment procedures (see, for example, European Patent Applications 0 455 482 and 0 451 611, which disclose methods for separating stem cells from a population of hematopoietic cells). Alternatively, unseparated hematopoietic cells and stem cell populations may be used as a target population for DNA transfer as described herein.

Compositions that Facilitate Use of Bifunctional Nucleic Acid Molecules

Yet another aspect of the invention features compositions including the molecules of the invention and another substance (or substances) that can facilitate the use of bifunctional nucleic acid molecules to influence a cell's biologic function. Such compositions can, for example, include substances that facilitate the uptake of exogenous nucleic acids by enhancing endocytosis, phagocytosis, potocytosis, micropinocytosis, and/or macropinocytosis (uptake enhancing agents). Uptake enhancing agents can include, for example, cytokines, e.g. M-CSF, PDGF, EGF, and HGF, as well as components of bacteria such as Shigella and Salmonella species. Other examples of uptake enhancing agents can include substances used in conventional transfection methods, such as transfection mediated by calcium phosphate.

Other examples of substances that can facilitate the use of a bifunctional nucleic acid molecule to introduce a biological effector sequence into a cell include substances that allow the internalized nucleic acid to escape from a subcellular vesicle (“escape agents”). Nucleic acid molecules labeled for visualization, e.g. fluorescently labeled, appear in a punctate pattern if they are contained in subcellular vesicles. According to the invention, a substance is an escape agent if, when admixed with a cells and labeled nucleic acid molecules, the molecules can form a less punctate and more diffuse pattern than if the substance is omitted from the mixture. A substance can also be an escape agent according to the invention if, when admixed with cells and a nucleic acid template for a polypeptide or a polynucleotide, expression of the encoded molecule by the cell is at least 20% greater than if the substance is omitted.

Escape agents can include, e.g., adenovirus particles (Curiel et al. (1991) Proc. Natl. Acad. Sci. USA 88:8850; Christen et al. (1993) Proc. Natl. Acad. Sci USA 90:2122-2126), portions of diphtheria toxin, including the transmembrane portion (Fisher and Wilson (1997) Biochem J321 :49-58), perfringolysin O (Gottschalk et al (1995) Gene Ther 2:498-503), peptides comprising sequences derived from influenza hemagglutinin (e.g., Plank et al (1994) J Biol Chem 269:12918-24), chloroquine (Guy et al (1995) Mol Biotechnol 3:237-48), and glycerol (Zauner et al (1997) Exp Cell Res 232:137-45). Another type of escape agent is an activator of protein kinase C, such as tetradecanoylphorbol 12,13-acetate or 1,2-dioctanoylglycerol (Reston et al (1991) Biochim Biophys Acta 1088:270-76). Other examples of escape agents include listeriolysin O, various phospholipases, and nuclease inhibitors.

Yet another example of a type of substance that can facilitate the use of bifunctional nucleic acid molecules to introduce a biological effector sequence into cells can include pharmaceutically/pharmacologically acceptable (i.e., applicable to mixing with pharmaceutical agents forth vitro testing such as for example for ex vivo uses which may require further testing or processing for use in mammals or humans), physiologically compatible (i.e., applicable for injection into an animal, particularly a human), and biologically compatible (i.e., acceptable for in vitro use on live cells but not necessarily acceptable, although not excluding acceptability, for in vivo use) carriers.

Bifunctional nucleic acids according to the invention may be delivered to cells using additional cell transfection enhancing agents, such as lipids (e.g., cationic lipids), peptides (e.g., cationic peptides) or synthetic polymers, for example, soluble DNA/polylysine complexes can be generated (Li et al., Biochem. J. 12, 1763 (1973)). Polylysine complexes tagged with asialoglycoprotein have been used to target DNA to hepatocytes in vitro (Wu and Wu, J. Biol. Chem. 262,4429(1987); U.S. Pat. No. 5,166,320). Lactosylated polylysine (Midoux et al. (1993) Nuc. Acids Res. 21,871-878) and galactosylated histones (Chen et al. (1994) Human Gene Therapy 5,429-435) have been used to target plasmid DNA to cells bearing lectin receptors, and insulin conjugated to polylysine (Rosenkrantz et al. (1992) Exp. Cell Res. 199,323-329) to cells bearing insulin receptors. Monoclonal antibodies have been used to target DNA to particular cell types (Machy et al. (1988) Proc. Natl. Acad. Sci. USA 85,8027-8031; Trubetskoy et al. (1992) Bioconjugate Chem. 3,323-27 and WO 91/17773 and WO 92/19287).

Peptides derived from the amino acid sequences of viral envelope proteins have been used in gene transfer when coadministered with polylysine DNA complexes (Plank et al. (1994) J. Biol. Chem. 269, 12918-24). Trubetskoy et al. (ibid) and Mack et al. ((1994) Am. J. Med. Sci. 102, 138-143) suggest that cocondensation of polylysine conjugates with cationic lipids can lead to improvement in gene transfer efficiency. WO 95/02698 used viral components to attempt to increase the efficiency of cationic lipid gene transfer.

### Use of the Invention

In another aspect, the invention pertains to methods of introducing nucleic acid molecules into cells. Such methods can apply, for example, to prokaryotic or eukaryotic cells in vitro or in vivo. As used herein, “in vitro” refers to circumstances in which cells are harbored and/or cultivated under artificial conditions. “In vivo” refers to circumstances in which cells are located on or within an organism. In vitro methods of the invention can include admixture of compositions of the invention with cells into which the introduction of a biological effector sequence is desired. Such methods can include the use of substances that can facilitate the use of bifunctional nucleic acid molecules to introduce a biological effector sequence into cells, e.g. substances that can be included in compositions of the invention. Methods of the invention can include uses of such substances other than inclusion in a composition of the invention, for example application of the substance to cells prior to or following application of a composition of the invention.

The bifunctional nucleic acid molecule can be admixed with a suitable buffer, e.g. TrisEDTA or phosphate-buffered saline. Typically, 0.1 ug-100 ug of bifunctional nucleic acid molecule in buffer is admixed with 103-107 cells that bear the target of the bifunctional nucleic acid molecule's aptamer.

In some methods of the invention, bifunctional nucleic acid molecules are delivered to a cell via receptor-mediated transfer in combination with conventional transfection methods to achieve a synergistic effect. Examples of such methods follow:
1. Transfection mediated by DEAE-dextran: Naked nucleic acid can be introduced into cells by forming a mixture of the nucleic acid and DEAE-dextran and incubating the mixture with the cells. A dimethylsulfoxide or chloroquine shock step can be added to increase the amount of nucleic acid uptake. DEAE-dextran transfection is only applicable to in vitro modification of cells and can be used to introduce nucleic acid transiently into cells but is not preferred for creating stably transfected cells. Thus, this method can be used for short term production of a gene product but is not a method of choice for long-term production of a gene product. Protocols for DEAE-dextranmediated transfection can be found in Current Protocols in Molecular Biology, Ausubel, F. M. et al. (e's.) Greene Publishing Associates, (1989), Section 9.2 and in Molecular Cloning: A Laboratory Manual. 2nd Edition. Sambrook et al. Cold Spring Harbor Laboratory Press, (1989), Sections 16.41-16.46 or other standard laboratory manuals.
2. Electroporation: Naked nucleic acid can also be introduced into cells by incubating the cells and the nucleic acid together in an appropriate buffer and subjecting the cells to a high-voltage electric pulse. The efficiency with which nucleic acid is introduced into cells by electroporation is influenced by the strength of the applied field, the length of the electric pulse, the temperature, the conformation and concentration of the nucleic acid and the ionic composition of the media. Electroporation can be used to stably (or transiently) transfect a wide variety of cell types and is only applicable to in vitro modification of cells. Protocols for electroporating cells can be found in Current Protocols in Molecular Biology, Ausubel, F. M. et al. (e's.) Greene Publishing Associates, (1989), Section 9.3 and in Molecular Cloning: A Laboratory Manual, 2nd Edition, Sambrook et al. Cold Spring Harbor Laboratory Press, (1989), Sections 16.54-16.55 or other standard laboratory manuals.
3. Liposome-mediated transfection (“lipofection”): Naked nucleic acid can be introduced into cells by mixing the nucleic acid with a liposome suspension containing cationic lipids. The nucleic acid/liposome complex is then incubated with cells. Liposome mediated transfection can be used to stably (or transiently) transfect cells in culture in vitro. Protocols can be found in Current Protocols in Molecular Biology, Ausubel, F. M. et al. (e's.) Greene Publishing Associates, (1989), Section 9.4 and other standard laboratory manuals. Additionally, gene delivery in vivo has been accomplished using liposomes. See for example Nicolau et al. (1987) Meth. Enz. 149:157-176; Wang and Huang (1987) Proc. NatL Acad Sci. SA 84:7851-785S; Brigham et al. (1989) Am. J. Med. Sci. 298:278; and Gould-Fogerite et al. (1989) Gene 84:429-438.
4. Transfection mediated by CaPO4: Naked nucleic acid can be introduced into cells by forming a precipitate containing the nucleic acid and calcium phosphate. For example, a HEPESbuffered saline solution can be mixed with a solution containing calcium chloride and nucleic acid to form a precipitate and the precipitate is then incubated with cells. A glycerol or dimethyl sulfoxide shock step can be added to increase the amount of nucleic acid taken up by certain cells. CaPO4-mediated transfection can be used to stably (or transiently) transfect cells and is only applicable to in vitro modification of cells. Protocols for CaPO4-mediated transfection can be found in Current Protocols in Molecular Biology, Ausubel, F. M. et al. (eds.) Greene Publishing Associates, (1989), Section 9.1 and in Molecular Cloning: A Laboratory Manual. 2nd Edition. Sambrook et al. Cold Spring Harbor Laboratory Press, (1989), Sections 16.32-16.40 or other standard laboratory manuals.

Generally, when naked nucleic acid is introduced into cells in culture (e.g., by one of the transfection techniques described above) only a small fraction of cells (about 1 out of 10(5)) typically integrate the transfected nucleic acid into theft genomes (i.e., the nucleic acid is maintained in the cell episomally). Thus, in order to identify cells which have taken up exogenous nucleic acid, it is advantageous to transfect nucleic acid encoding a selectable marker into the cell along with the nucleic acid(s) of interest. Preferred selectable markers include those which confer resistance to drugs such as G418, hygromycin and methotrexate. Selectable markers may be introduced on the same nucleic acid molecule as the gene(s) of interest or may be introduced on a separate molecule.

In vivo methods of the invention can include methods of administration of a composition of the invention to an organism that harbors cells into which introduction of a biological effector sequence is desired. The organism can be any plant or animal, but in a preferred embodiment is chosen from among the following: a human, a domestic animal, a cultivated plant. The targeted cells can be integral to the recipient organism or can, for example, be commensal microbes harbored by that organism. Routes of administration can include, e.g., intravenous, intraarterial, intrathecal, intraperitoneal, subcutaneous, intradermal, epicutaneous, oral, ocular, mucosal, or aerosol.

According to the invention, internalization of a nucleic acid by a cell can be detected in several ways. For example, if the nucleic acid encodes a polypeptide or a polynucleotide, internalization can be indicated by an increase in synthesis of the encoded molecule by the cell. If the nucleic acid encodes or comprises an antisense sequence, a ribozyme, or a nucleic acid ligand, internalization can be indicated by a decrease in net synthesis of the target molecule by the cell or a decrease in activity of the target molecule.

In another method for detecting internalization of a nucleic acid by a cell, the nucleic acid is conjugated to a detectable marker, e.g. a radioactive marker, an enzyme such as alkaline phosphatase or horseradish peroxidase, or a fluorochrome such as fluorescein isothiocyanate or phycoerythrin. Methods for effecting such conjugation are well known to those skilled in the art. For example, the nucleic acid can be botinylated and admixed with a streptavidin-linked marker. The conjugates are then admixed with the cells according to a method of the invention, extracellular nucleic acid removed, e.g. by denaturation or exonuclease treatment and washing, and the marker detected. If the nucleic acid is internalized, marker uptake will be at least 20% greater than when marker alone is used in the assay. The aptamer and biological effector of a bifunctional nucleic acid molecule can be detected separately in such an assay if each is conjugated to a different marker, e.g. fluorochrome with different spectral characteristics.

Dosage, Mode of Administration, and Pharmaceutical Formulations

In the methods of the invention, a composition of the invention is administered in vivo or applied in vitro in such a manner and amount and on such a schedule that a biological effector sequence is introduced into an appropriate number and type of target cells such that the desired effect is achieved. The specific conditions of a method can depend, for example, on the aptamer and the biological effector sequence, the nature of the target cells, and the effect desired.

Bifunctional nucleic acid molecules described herein may be prepared as injectables, either as liquid solutions or suspensions; solid forms suitable for solution in or suspension in, liquid prior to infection can also be prepared. The preparation can also be emulsified, or the bifunctional nucleic acid molecules encapsulated in liposomes. The active ingredients are often mixed with carriers which are pharmaceutically acceptable and compatible with the active ingredient. The term “pharmaceutically acceptable carrier” refers to a carrier that does not cause an allergic reaction or other untoward effect in subjects to whom it is administered. A “biologically compatible carrier” is one which is compatible with in vitro cellular transfection. Suitable pharmaceutically acceptable carriers include, for example, one or more of water, saline, phosphate buffered saline, dextrose, glycerol, ethanol, or the like and combinations thereof. In addition, if desired, the formulation can contain minor amounts of auxiliary substances such as wetting or emulsifying agents, and/or pH buffering agents.

Bifunctional nucleic acid molecules of the invention can be administered parenterally, by injection, for example, either subcutaneously or intramuscularly. Additional formulations which are suitable for other modes of administration include suppositories, and in some cases, oral formulations or formulations suitable for distribution as aerosols. For suppositories, traditional binders and carriers may include, for example, polyalkylene glycols or triglycerides; such suppositories may be formed from mixtures containing the active ingredient in the range of 0.5% to 10%, preferably 1%-2%. Oral formulations include such normally employed excipients as, for example, pharmaceutical grades of mannitol, lactose, starch magnesium stearate, sodium saccharine, cellulose, magnesium carbonate, and the like. These compositions take the form of solutions, suspensions, tablets, pills, capsules, sustained release formulations or powders and contain 10%-95% of active ingredient, preferably 25-70%.

The bifunctional nucleic acid molecules of the invention can be formulated into the compositions as neutral or salt forms. Pharmaceutically acceptable salts include the acid addition salts (formed with free amino groups of the peptide) and which are formed with inorganic acids such as, for example, hydrochloric or phosphoric acids, or with organic acids such as acetic, oxalic, tartaric, maleic, and the like. Salts formed with the free carboxyl groups can also be derived from inorganic bases such as, for example, sodium, potassium, ammonium, calcium, or ferric hydroides, and such organic bases as isopropylamine, trimethylamine, 2-ethylamino ethanol, histidine, procaine, and the like.

The bifunctional nucleic acid molecules are administered in a manner compatible with the dosage formulation, and in such amount as will be prophylactically and/or therapeutically effective. The quantity to be administered depends on the subject to be treated, including, e.g., the subject's body mass. Precise amounts of active ingredient required to be administered depend on the judgment of the practitioner and may be peculiar to each subject. It will be apparent to those of skill in the art that the therapeutically effective amount of bifunctional nucleic acid molecules of this invention will depend, inter alia, upon the administration schedule, the unit dose of aptamer and biological effectors sequence administered, whether the bifunctional nucleic acid molecules are administered in combination with other agents, the health of the recipient, and the therapeutic activity of the particular bifunctional nucleic acid molecule. In general, though, the dosage required for therapeutic efficacy will range from about 0.1 ug to 20 mg nucleic acid/kg body mass/dose.

Cells transfected with the bifunctional nucleic acid molecules of the invention can subsequently be administered to a subject to achieve a therapeutic effect, e.g. to provide a polypeptide which is encoded by the biological effector sequence and in which the subject is deficient. Typically, the cells will be formulated as a suspension in a pharmaceutically acceptable carrier, e.g. a physiologically compatible buffer. While the number of cells required for a therapeutic effect will vary according to both subject and cell characteristics, it will generally be between about 103 and 109 cells/dose.

### Exemplification

### EXAMPLE 1

In one example, a cloning vector comprising a bifunctional nucleic acid molecule-encoding sequence is prepared in the following manner: The following oligodeoxynueleotides are synthesized: 1) 5′AACGGCCGCGGCTAGTCCACACACAGAACCGTT3′(SEQ ID NO: 1), the sense strand encoding a vascular endothelial growth factor-binding RNA (Jellinek et al, Biochemistry 1994 33:10450-10456) and 2) the complementary strand 5′AACGGTTCTGTGTGTGTGGACTAGCCGCGGCCGTTTCGA3′ (SEQ ID NO: 2) with an additional 3′ Hind Ill compatible sequence. These oligonucleotides are admixed and annealed to each other. A nucleotide sequence including the E. coli beta galactosidase gene is prepared by digesting the pUC19 plasmid (Genbank accession number X02514) with Nar I and Hind III, and isolating the resulting 212 base pair fragment by agarose gel electrophoresis and elution. The annealed oligonucleotide and the pUC 19-derived fragment are ligated using T4 DNA ligase. The resulting molecule is ligated to pSP7O plasmid (Promega, Madison, Wis.) that has been digested with Cla I and Xho I, exploiting the complementarity of Nar I and Cla I overhangs. The free Xho I end of the plasmid is blunted with Klenow and the plasmid is circularized with T4 DNA ligase. An RNA bifunctional nucleic acid molecule is obtained by subjecting the plasmid to standard in vitro transcription procedures using SP6 RNA polymerase.

### EXAMPLE 2

In another example, the following oligodeoxynucleotides are synthesized:

1)) 5′CGCGAACGGCCGCGGCTAGTCCACACACAGAACCGTT3′ (SEQ ID NO: 3), the sense strand encoding a vascular endothelial growth factor-binding RNA (Jellinek et al, Biochemistry 1994 33:10450-10456) with an additional 5′ Hae II compatible site and 2) the complementary strand 5′AACGGTTCTGTGTGTGTGGACTAGCCGCGGCCGTTTCGA3′ (SEQ ID NO: 4). These oligonucleotides are admixed and annealed to each other. A nucleotide sequence including the E. coli beta galactosidase gene is prepared by digesting the pUC19 plasmid (Genbank accession number X02514) with Hae I and Hind III, and isolating the resulting 212 base pair fragment by agarose gel electrophoresis and elution. The annealed oligonucleotide and the pUC 19-derived fragment are ligated using T4 DNA ligase. The resulting molecule is ligated to pSP7O plasmid (Promega) that has been digested with Hind III and Bgl II. The free Bgl II end of the plasmid is blunted with Klenow and the plasmid is circularized with T4 DNA ligase. An RNA bifunctional nucleic acid molecule is obtained by subjecting the plasmid to standard in vitro transcription procedures using SP6 RNA polymerase.

### EXAMPLE 3

In an example of a method of the invention, the bifunctional nucleic acid molecules of Example 1 or Example 2 is introduced into CMS5 mouse fibrosarcoma cells that have been engineered to express a recombinant fusion polypeptide consisting of the intracellular and transmembrane portions of a fibroblast growth factor receptor (Genbank Ace. No. M34185) fused in-frame to the human VEGF 165 protein (see Swiss-Prot P15692), with the VEGF portion oriented extracellularly. About 0.1-100 ug of the bifunctional nucleic acid molecule is admixed in a suitable buffer with about 103-107 of these cells in vitro. Subsequent expression of beta galactosidase is determined by X-gal staining.

### EXAMPLE 4

A library of RNA's, each having 32 randomized bases between two fixed sequences of 30-45 bases, is constructed. An aptamer for the transferrin receptor (Genbank Ace. No. M11507) is isolated by SELEX, using the receptor's extracellular domain as a target. An antisense oligodeoxynucleotide which binds to the template region of the RNA component of human telomerase (5′TAGGGTTAG3′) (Feng et al (1995) Science 269:1236-41) is synthesized and treated with calf alkaline phosphatase. The aptamer and the antisense biological effector sequence are ligated using T4 RNA ligase.

The resulting bifunctional nucleic acid is useful for treating malignant tumors. The bifunctional nucleic acid binds to transferrin receptor-bearing cells and delivers to the cell antisense oligonucleotide that binds to the RNA component of human telomerase, thus inhibiting function of telomerase enzyme.

### EXAMPLE 5

The aptamer of Example 4 is synthesized with an additional 5′GGGGGGGCCCCCCC3′ (SEQ ID NO: 5) at the 5′ end and an additional 5′AAAAAAAUUUUUUU3′ (SEQ ID NO: 6) at the 3′ end. The antisense sequence of Example 4 is synthesized with an additional 5′AAAAAAAUUUUUUU3′ (SEQ ID NO: 6) at the 3′ end. The molecules are admixed in a molar ratio of 8 aptamers: 1 antisense for annealing into concatemers.

The resulting bifunctional nucleic acid is useful for treating malignant tumors.

### EXAMPLE 6

A phosphorothioate antisense oligodeoxynucleotide designed to inhibit expression of the reporter protein Enhanced Green Fluorescent Protein (EGFP) was Watson-Crick hybridized to an aptamer with high affinity for the cell surface receptor human L-selectin. This nucleic acid molecule was then incubated with Jurkat cells, a human leukemic T-cell line which naturally expresses human L-selectin, and which, for the purpose of this experiment, had been stably transfected with the pEGFP-n2 expression plasmid encoding EGFP (Clohtech Laboratories, Palo Alto, Calif.). Cells were then analyzed by flow cytometry to determine fluorescence intensities after incubation with aptamer-antisense nucleic acid molecules or with antisense alone.

For this experiment a 32 base mixed-backbone oligodeoxynucleotide with a sequence of: 5′TGGTACCACTCGTTCCCGGATGGATGCTAGAC 3′ (SEQ ID NO: 7) was purchased from Synthegen, LLC (Houston, Tex.) and purified by reverse-phase HPLC. The first 18 bases at the 5′ end were Watson-Crick complementary to a region of the pEGFP-n2 plasmid starting three bases before the start codon of the EGFP gene and were linked by phosphorothioate bonds. The final 14 bases at the 3 ‘end of the oligodeoxynucleotide had a phosphodiester backbone and were Watson-Crick complementary to the 5’ end of a 79-base single stranded phosphodiester aptamer with high affinity and specificity for human L-selectin (SEQ ID #134 of Parma et al, PCT Publication WO 96/40703). The sequence of this aptamer was 5′CTACCTACGATCTGACTAGCCGGACATGAGCGTTACAAGGTGCTAAACGTAACGT TGCTTACTCTATGTAGTTCC3′ (SEQ ID NO: 8) (purchased from Midland Certified Reagent Co. Midland, Tex., where it was purified by trityl-selective perfusion HPLC).

The antisense and aptamer molecules were simultaneously added in a 1:1 molar ratio to 2×10⁶ EGFP-expressing Jurkat cells in 500 μL of a buffer consisting of 1 mM CaCl₂, 1 mM MgCl₂, 125 mM NaCl, 5 mM KCl, and 20 mM HEPES, pH 7.4 and incubation proceeded for 30 minutes at room temperature. 250 μl of each suspension was diluted into duplicate tubes containing 750 μL of RPMI 1640 media with 10% FCS and 100 μM chloroquine.

Immediately after suspension in media, 250 μL was removed and cells were centrifuged and washed twice in PBS buffer, suspended in 500 μL PBS, and stained with propidium iodide for baseline fluorescence and viability analysis.

The remaining 750 μL of cells and nucleic acid molecules were then incubated for 42 hours at 37° C. with 95% O₂, 5% CO₂. Then another 250 μL sample was drawn from each sample. These cells were then prepared and analyzed for fluorescence as at time zero.

The results of this experiment indicate that the aptamer-antisense conjugated molecules exerted a greater antisense effect than antisense molecules alone. FACScan analyses of viable (determined by propidium iodide exclusion) Jurkat cells showed that, at 42 h, mean cell fluorescence of cells incubated in a 0.5 μM concentration of antisense alone was 13.4% below that of appropriate control cells incubated with neither aptamer nor antisense. The mean fluorescence of cells incubated with 0.5 μM of the aptamer-antisense molecule was 25.7% below that of appropriate controls incubated with 0.5 μM of aptamer alone, so that at this concentration the aptamer nearly doubled the effect of the antisense. Cells incubated in a 1 μM concentration of the aptamer-antisense combination had a mean fluorescence 23.7% below that of appropriate controls, while the mean fluorescence of cells incubated with antisense alone was only 18.3% below that of appropriate controls. Mean fluorescences of the two types of controls were comparable, differing much less than 1%.

### EXAMPLE 7

The aptamer and first 18 bases 5′ of the antisense described in Example 6 are adjoined by a covalent phosphodiester bond. This bifunctional molecule is a 98 base single stranded oligodeoxynucleotide which has a sequence of:

| | |
|---|---|
| 5′CTACCTACGATCTGACTAGCCGGACATGAGCGTT | (SEQ ID NO: 9) |
| ACAAGGTGCTAAACGTAACGTTGCTTACTCTATGTA | |
| GTTCCTGGTACCACTCGTTCCCG3′. | |

### EXAMPLE 8

A bifunctional nucleic acid is constructed using the aptamer of Example 6 and an antisense molecule consisting of the 14 3′ bases of the antisense molecule of Example 6 appended 3′ to the phosphorothioate antisense molecule GEM 91, which inhibits the expression of HIV gag and is described in Veal et al, 1998, Antiviral Research 38:63-73. Aptamer and antisense are annealed in PBS at room temperature. The bifunctional nucleic acid molecule can target the antisense to T cells and is useful for treating HIV infection.

### EXAMPLE 9

The reporter gene pEGFP-n2 from Clontech Laboratories is linearized by digestion with the restriction enzyme ApaLI in a non-coding region leaving a 5′ sense overhang with the sequence ACGT on one end and an identical overhang on the 5′antisense strand. The following oligodeoxynucleotides are synthesized: 1) 5′TGCAGGGGGGGGGGAACTACATGAGAG3′ (SEQ ID NO: 10), which has the first 4 bases at 5′ end complementary to the ApaLI overhang, an internal 8 bases which are complementary to the oligomer #2, and the final 15 bases which are Watson-Crick complementary to the 5′ end of a 79 base human L-Selectin aptamer,#3, 2) 5′CCCCCCCC3′ which is complementary to 8 bases internal in oligomer #1, and 3) 5′CTACCTACGATCTGACTAGCCGGACATGAGCGTTACAAGGTGCTAAACGTAACGT TGCTTACTCTATGTAGTTCC 3′ (SEQ ID NO: 11) which is a high affinity aptamer for human L-selectin (SEQ ID #134, Parma et al, PCT Publication WO 96/40703) The first two oligodeoxynucleotides are annealed together by room temperature incubation and then are ligated to the linearized pEGFP-n2 plasmid with T4 DNA ligase. The linearized pEGFP-n2 plasmid now has two linkers ligated to both of its ends which are complementary to a 15 base region of the 5′end of the human L-selectin aptamer. The pEGFP-n2 plasmid is incubated with the aptamer at a 1:2 molar ratio in a buffer consisting of 1 mM CaCl₂, 1 mM MgCl₂, 125 mM NaCl, 5 mM KCl, and 20 mM HEPES, pH 7.4 for 30 minutes. Wild type Jurkat cells at a concentration of 5×10⁵ which endogenously express human L-selectin are then added to the plasmid and aptamer mix, and incubation continues for another 30 minutes. The mix is then equally divided and diluted into duplicate tubes containing RPMI 1640 with 10% FCS and 100 μM chloroquine. After 72 hours cells which have been stably transfected with pEGFP are selected for Neomyein resistance by addition of 1 mg/mL of G41 8 antibiotic. Only cells which have stably integrated the neomycin resistance gene included in the pEGFP-n2 plasmid will survive.

A control group consists of cells which receive only linkers ligated to plasmid in a 2:1 molar ratio. For each group of cells a range of final pEGFP-n2 concentrations of 0 through 500 pM up to 1 μM is used. The aptamer-plasmid molecule will yield a transfection efficiency at least 20% higher than that of plasmid alone, as measured by number of selected clones.

### EXAMPLE 10

A variation of example 6 would entail using the same human L-selectin aptamer oligodeoxynucleotide (SEQ ID #134, Parma et al, PCT Publication WO 96/40703) with a sequence of: 5′CTACCTACGATCTGACTAGCCGGACATGAGCGTTACAAGGTGCTAAACGTAACGT TGCTTACTCTATGTAGTTCC3′ (SEQ ID NO: 11) in conjunction with three distinct mixed backbone antisense molecules. Each antisense molecule will retain the 18 phosphorothioate bases which are complementary to the EGFP start codon region, and will have at least one end with 14 phophodiester bases complementary to the aptamer molecule. Antisense 1 (A1) has the sequence: 5′TGGTACCACTCGTTCCCGGATGGATGCTAGAC3′ (SEQ ID NO: 7) which is identical to the antisense used in example 6 and consists of an EGFP hybridization region in the first 18 bases of the 5′ end and an aptamer hybridization region at the 14 bases of the 3′ end. A2 has the sequence 5′AGAGTACATCAAGGTGGTACCACTCGTTCCCG3′ (SEQ ID NO: 12) in which the first 14 bases at the 5′ end are complementary to the first 14 bases at the 3′ end of the aptamer molecule, and the last 18 bases at the 3′ end comprise the EGFP hybridization region. The A3 molecule contains aptamer hybridization regions at both the 5′ and 3′ region and maintains the EGFP antisense sequence in the internal 18 phosphorothioate base pairs:

| | |
|---|---|
| 5′AGAGTACATCAAGGTGGTACCACTCGTTCCCG | (SEQ ID NO: 13) |
| GATGGATGCTAGAC3′. | |

By varying the molar ratio of A1 and A2 to A3, it is possible to regulate the length of a concatamerized aptamer-antisense chain. For example, aptamer, A1, A2, and A3 can be admixed in a molar ratio 3:1:1:2, so that the nucleic acid molecules thus formed will on average comprise three aptamer units to four antisense units. These molecules can then be used in the method otherwise described in Example 6.

### EQUIVALENTS

Those skilled in the art will recognize, or will be able to ascertain using no more than routine experimentation, many equivalents of the specific embodiments of the invention described herein. Such equivalents are intended to be encompassed by the following claims.

1. A nucleic acid molecule comprising:
a first nucleic acid sequence comprising an aptamer which binds to a cell surface molecule, covalently linked to a second nucleic acid sequence comprising a biological effector sequence, wherein said biological effector sequence is not an aptamer.

2. A nucleic acid molecule comprising:
a first nucleic acid sequence comprising an aptamer which binds to a cell surface molecule, linked via Watson-Crick base pairing to a second nucleic acid sequence comprising a biological effector sequence, and wherein said biological effector sequence is not an aptamer.

3. The molecule of claim 1 or 2, further comprising a third nucleic acid sequence which is an aptamer that is covalently linked to said nucleic acid molecule.

4. The molecule of claim 1 or 2, further comprising a third nucleic acid sequence which is an aptamer that is linked via Watson-Crick base pairing to said nucleic acid molecule.

5. The molecule of claim 3 wherein said third nucleic acid sequence comprises an aptamer that is different from said first nucleic acid comprising an aptamer.

6. The molecule of claim 4 wherein said third nucleic acid sequence comprises an aptamer that is different from said first nucleic acid sequence comprises an aptamer.

7. The nucleic acid molecule of claim 1 or 2, comprising DNA or RNA.

8. The nucleic acid molecule of claim 1 or 2, wherein said biological effector sequence encodes a polypeptide or polynucleotide.

9. The nucleic acid molecule of claim 1 or 2, wherein said biological effector sequence comprises a messenger RNA.

10. The nucleic acid molecule of claim 8, wherein the coding sequence of said biological effector sequence comprises double-stranded DNA, and wherein said biological effector sequence comprises a promoter.

11. The nucleic acid molecule of claim 1 or 2, wherein said biological effector sequence comprises an antisense sequence.

12. The nucleic acid molecule of claim 1 or 2, wherein said biological effector sequence comprises a nucleic acid enzyme.

13. A nucleic acid molecule comprising a template for the assembly of the molecule of claim 1.

14. A cloning vector comprising the molecule of claim 1.

15. A cloning vector comprising the molecule of claim 11.

16. A composition comprising the molecule of claim 1 or 2 and a biologically acceptable carrier.

17. A composition comprising an admixture of a nucleic acid molecule of claim 1 or 2 and a cell in vitro that bears a target molecule for said aptamer.

18. A cell transfected in vitro with a nucleic acid molecule, wherein the nucleic acid molecule is chosen from the group consisting of the nucleic acid molecule of claim 1, the nucleic acid molecule of claim 2, the nucleic acid molecule of claim 13, the cloning vector of claim 14, and the cloning vector of claim 15.

19. A method of introducing a biological effector sequence into a cell comprising contacting the nucleic acid molecule of claim 1 or 2 in vitro with a host cell, wherein said aptamer of said nucleic acid molecule of claim 1 or 2 binds to a cell surface molecule of said host cell and permits the internalization of said biological effector sequence, whereby said biological effector sequence is internalized by said host cell.

20. A method of introducing a biological effector sequence into an organism, comprising:
introducing a biological effector sequence into a host cell by contacting the nucleic acid molecule of claim 1 or 2 in vitro with said host cell, wherein the aptamer of said nucleic acid molecule of claim 1 or 2 binds to a molecule on the surface of said host cell and permits the internalization of said biological effector sequence, wherein said biological effector sequence of said nucleic acid molecule of claim 1 or 2 is internalized by said host cell; and administering said host cell to the organism.
